(19) United States
(12) Patent Application Publication
(10) Pub. No.: US 2011/0306954 A1
(43) Pub. Date: Dec. 15, 2011

(54) PULSED LASER WITH AN OPTICAL FIBRE FOR HIGH-ENERGY SUB-PICOSECOND PULSES IN THE L BAND, AND LASER TOOL FOR EYE SURGERY

(75) Inventors: Franck Morin, Bures Sur Yvette (FR); Marc Hanna, Limours (FR); Frederic Druon, Orsay (FR); Patrick Georges, Noisy Le Roi (FR)

(73) Assignee: CENTRE NATIONAL DE LA RECHERCHE SCIENTIFIQUE, Paris cedex 16 (FR)

(21) Appl. No.: 13/139,801

(22) PCT Filed: Dec. 16, 2009
(86) PCT No.: PCT/FR09/52579

§ 371 Date: Jun. 15, 2011

(30) Foreign Application Priority Data
Dec. 17, 2008 (FR) 0858687

(51) Int. Cl. A61F 9/008; A61B 18/22

(52) U.S. Cl. 606/4

(57) ABSTRACT

A a pulsed laser (10) with an optical fibre and frequency shifting for amplifying high-energy sub-picosecond pulses, includes an oscillator (1), a fibre stretcher (3), one or more preamplification stages and one power amplification stage including an erbium doped or erbium-ytterbium codoped optical fibre section (4, 7), a pump (8) suitable for optically pumping by coupling in the optical fibre of the power amplifier (7) and a compressor (9). According to the invention, the pump (8) of the power stage generates at least one pump wavelength λP between 1530 and 1565 nm, the laser pulse emission wavelength (20) is between 1565 and 1625 nm and the energy of the laser pulses (20) is between 10 nJ and several dozen μJ. A tool for eye surgery including such a laser is also described.

The present invention relates to an optical-fiber pulsed laser suitable for generating ultra-short pulses of high energy at a high rate, and at an emission wavelength that lies in a transparency window of the cornea.

More precisely, the invention relates to a chirped pulse amplification fibre laser for amplifying high energy sub-picosecond pulses with an emission wavelength lying in the range 1565 nanometers (nm) to 1625 nm.

Such a laser serves to generate laser pulses of high energy, i.e., as used in this document, the term “high energy” is used to mean pulses presenting energy lying in the range 100 nanojoules (nJ) per pulse to 100 microjoules (μJ) per pulse, at a high repetition rate (10 kilohertz (kHz) to 1 megahertz (MHz)), and presenting good spatial quality in the output beam (close to a Gaussian beam, M²<1.5).

The laser of the invention is constituted essentially by optical fibers, thereby making it very robust and suitable for incorporation in devices that are compact.

The invention also relates to a tool for ophthalmic surgery using such a laser, in particular for deep cutting of the cornea or for treating glaucoma.

Lasers are beginning to be used for cutting the cornea instead of mechanical tools such as a microkeratome. The depth of cut may be as much as one millimeter and it must be controlled very accurately. In laser cutting operations, a series of laser pulses is focused along the desired line of cut. The patient's eye is prevented from moving throughout the surgery, so the surgery must therefore be as short as possible. The lasers used for cutting the cornea must therefore be suitable for generating pulses at a very high rate. In the application to cutting the cornea, it is essential to have pulses that are simultaneously of short duration, of high energy, of good spatial quality, and that are absorbed and/or diffused little by the healthy or diseased tissue in which the laser beam is focused. The quality of cutting depends on the spatial and temporal quality of the laser pulses, on their energy, and on their focusing. A laser makes it possible to perform cutting that is more accurate and more complex than can be performed with a microkeratome.

Nevertheless, existing lasers do not enable such cutting to be performed quickly since the energy per pulse is insufficient and/or because of the way the laser beam is diffused and/or deformed on passing through the optical media of a pathological eye, which media degrade the focusing of the beam.

Pulsed fiber lasers have been used in the fabrication of tools for medical or cosmetic treatment. Document U.S. Pat. No. 6,723,090 (Altshuler et al.) describes a fiber laser including a pump diode and a section of amplifier optical fiber. That low-energy laser is used in dermatological or medical treatment at a wavelength that is tunable so as to be absorbed by tissue. Such a laser may operate in triggered pulse mode. Nevertheless, the minimum duration of the pulses is not less than 10 microseconds (ms) and the repetition rate of the pulses is limited. Such a laser is not suitable for producing high-energy sub-picosecond pulses at a high repetition rate.

Elsewhere, optical fiber lasers capable of generating high-energy femtosecond pulses at high repetition rates have recently been developed. Fiber lasers based on chirped pulse amplifier (CPA) technology serve to limit the non-linear effects that appear while amplifying pulses in fibers, and thus to obtain pulses of high energy and short duration.

Document U.S. Pat. No. 7,131,968 (Bendett et al.) describes a fiber laser for ophthalmic surgery and more particularly for rapid cutting of the cornea in operations for correcting refraction. That laser is a chirped pulse amplification fiber laser comprising an oscillator, a fiber pulse stretcher, a pre-amplifier, a power amplifier, and a pulse compressor. That laser generates femtosecond pulses at a high repetition rate (50 kHz to 100 kHz). Like most ytterbium-doped fiber lasers, that device emits pulses at the wavelength of 1.05 micrometers (μm).

Unfortunately, edematous corneas diffuse strongly at wavelengths close to 1 μm. The energy of the laser pulses is thus no longer sufficient at the point of focus to perform effective cutting. Such lasers are not adapted to cutting pathological corneas rapidly, accurately, and reliably.

At present there exist no sub-picosecond lasers based on erbium-doped fibers that present sufficient energy for cutting the cornea and that operate in the wavelength range 1565 nm to 1625 nm.

Erbium-doped optical fiber amplifiers pumped with 980 nm or 1480 nm laser diodes do indeed present an emission band of 1565 nm to 1625 nm (known as L-band in telecommunications). Nevertheless, in L-band, the length of the amplifier optical fiber needs to be much longer than in band C (1530 nm to 1565 nm). Pulses that propagate over a long length of fiber are subjected to non-linear effects that deform those pulses in time. Furthermore, the spectral gain of a L-band amplifier pumped with a laser diode at 980 nm or 1480 nm varies strongly along the amplifier fiber. Such an amplifier does not enable high-energy sub-picosecond pulses to be generated in L-band.

An object of the present invention is to remedy those drawbacks, and the invention relates more particularly to a chirped pulse amplification fiber laser for amplifying sub-picosecond pulses of good spatial quality and high energy, the laser comprising an oscillator, a fiber pulse stretcher, at least one rate reducer, one or more pre-amplification stages, a power amplification stage, and a pulse compressor. The oscillator is suitable for emitting light pulses of sub-picosecond duration with energy lying in the range 10 picojoules (pJ) to 10 nJ with an emission wavelength lying in the range 1565 nm to 1625 nm. The pulse stretcher is suitable for stretching those light pulses in time. Each pre-amplification stage comprises a section of erbium-doped or erbium-ytterbium co-doped optical fiber and a pump suitable for optically pumping the section of pre-amplification optical fiber. The rate reducer(s) is/are suitable for reducing the repetition rate of the output laser pulses to lie in the range 10 kHz to 1 MHz. The power amplification stage likewise comprises a section of erbium-doped or erbium-ytterbium co-doped optical fiber and a pump suitable for optically pumping said section of optical fiber. At the output from the power amplifier stage, a compressor is suitable for recompressing the amplified light pulses in time. According to the invention, the power amplifier pump generates at least one pump wavelength (λ_{P}) in the wavelength band of 1530 nm to 1565 nm so as to obtain laser output pulses of emission wavelength lying in the range 1565 nm to 1625 nm and of energy lying in the range 100 nJ to 100 μJ.

In an embodiment of the invention, the section of optical fiber of the power amplifier stage is a large mode area (LMA) fiber.

In a particular embodiment of the invention, the output beam has good spatial quality, with a coefficient M² less than 1.5.

In a particular embodiment of the invention, the pump of the power amplifier is an erbium-doped or erbium-ytterbium co-doped optical fiber laser.

In a particular embodiment of the invention, the pump of the power amplifier is directly coupled into the doped core of the power amplifier optical fiber.

In a particular embodiment of the invention, the pump of the power amplifier is suitable for pumping the section of power amplifier optical fiber in co-propagation and/or in contra-propagation manner.

In a particular embodiment of the invention, the pump of the power amplifier is suitable for generating at least one second pump wavelength (λ_{P}′) lying in the range 1530 nm to 1565 nm.

In a particular embodiment of the invention, the wavelength (λ_{P}) of the pump of the power amplifier is adjustable so as to optimize the output pulses spectrally and temporally.

In a particular embodiment of the invention, the pump of the power amplifier is an optical fiber laser.

In a particular embodiment of the invention, the laser includes another pump suitable for optically pumping the section of power amplifier optical fiber at another pump wavelength λ′_{P} lying in the range 970 nm to 990 nm simultaneously with the first pump at the pump wavelength λ_{P} so as to increase the amplification gain in the section of amplifier optical fiber.

The invention also provides a laser tool for ophthalmic surgery, the tool comprising an optical fiber pulsed laser according to any of the embodiments described. In a particular embodiment of the laser tool of the invention for ophthalmic surgery, the energy of the laser pulses is greater than 100 nJ.

The above-specified characteristics of such a laser make it possible to have a laser that generates sub-picosecond laser pulses of energy lying in the range 100 nJ to 100 μJ and of wavelength lying in the range 1565 nm to 1625 nm.

A particularly advantageous first application of the L-band, high-energy and sub-picosecond pulsed laser of the invention lies in a laser tool for ophthalmic surgery for deep cutting of the cornea in the human or animal eye.

Below, the term “sub-picosecond” is used for pulses of duration that is generally less than one picosecond, and that may extend up to 1 or 2 picoseconds. The term “femtosecond pulses” is used to mean pulses of duration lying in the range 1 femtosecond to several hundred femtoseconds.

The present invention also relates to characteristics that appear from the following description and that should be considered in isolation or in any technically feasible combination.

This description that is given by way of non-limiting example serves to make it better understood how the invention can be performed, and is given with reference to the accompanying drawings, in which:

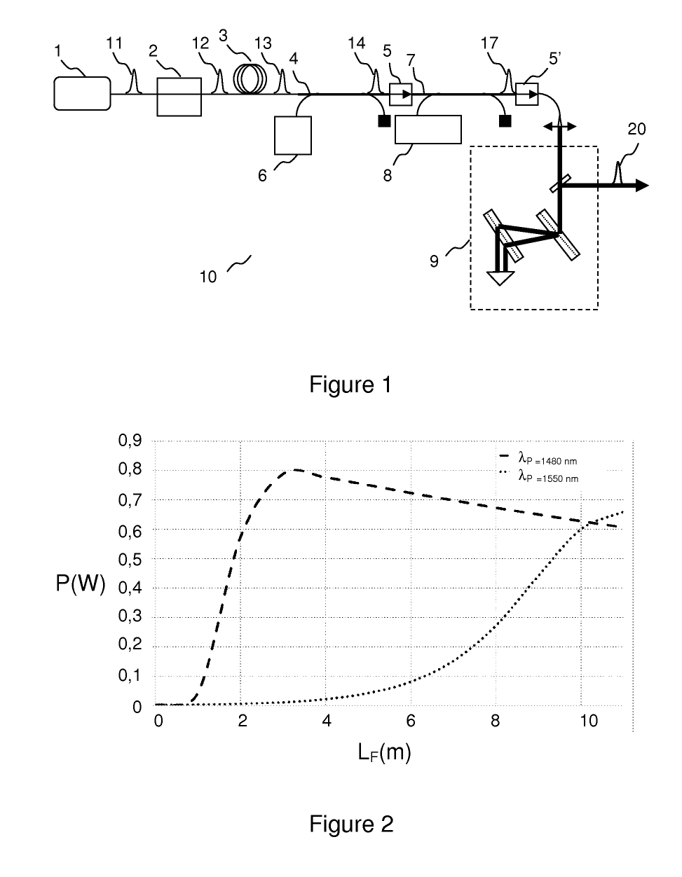

FIG. 1 shows an embodiment of a device of the invention;

FIG. 2 plots mean power curves as a function of amplifying fiber length for two pump wavelengths (λ_{P}); and

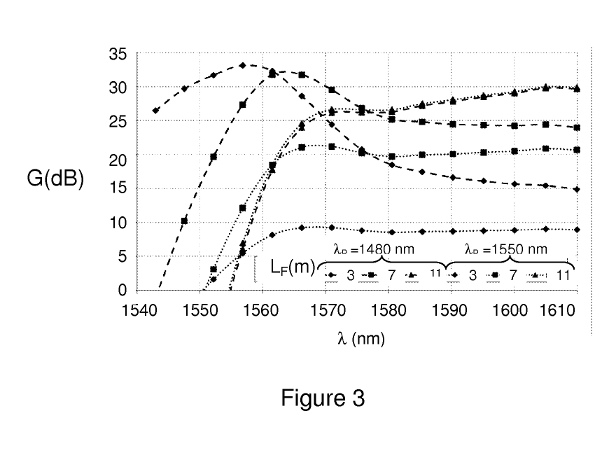

FIG. 3 plots curves of spectral gain (α) for various pump wavelengths (λ_{P}) and fiber lengths (L_{F}) of the power amplifier.

In the preferred embodiment shown diagrammatically in FIG. 1, the pulsed laser 10 of the invention is made up of a plurality of portions: an oscillator 1 producing sub-picosecond pulses; a rate reducer 2 of the acousto-optical or electro-optical modulator type; a pulse stretcher 3; an optical fiber preamplifier stage 4; an optical fiber power amplifier stage 7; and a pulse compressor 9.

The laser 10 is essentially constituted by fiber components, in which the active portion is an Er-doped fiber or an Er—Yb co-doped fiber. The oscillator 1 emits light pulses 11 of sub-picosecond duration with a center wavelength lying in the range 1565 nm to 1625 nm. The dispersion-compensation and mode-locking functions are performed in waveguide optics. Mode locking may be active, using electro-optical modulators, or passive, e.g. using the non-linear polarization rotation effect or a saturable absorbent Bragg mirror.

The rate reducer 2 is constituted by an electro-optical or acousto-optical modulator, which may be solid (beam in free space) or integrated and connected to optical fibers. The rate reducer 2 serves to adjust the repetition rate of the pulses depending on the task to be undertaken. The rate and/or the number of pulses per burst are optimized depending on the type of application.

The stretcher 3 is a dispersive system that may be implemented using a highly normal dispersion compensating fiber (DCF) with a length that is adjusted to obtain the desired stretching. The stretcher may also be implemented using a combination of solid optical components such as prisms and diffraction gratings together with lenses and mirrors for providing an optical imaging system. The system serves to lengthen the duration of the initial pulses 11 so as to limit non-linear effects in the pre-amplification and power amplification stages.

The pre-amplification and power amplification stages are based on respective fibers 4 and 7 that are erbium-doped or Er—Yb co-doped. The pre-amplification stages may be pumped by monoemitter diodes 6 coupled to the monomode core of the amplifying fiber 4. The device also includes optical isolators 5, 5′. The power stage presents the main original feature of the invention and it is described below.

The compressor 9 is a dispersive device performing (to first order) the dispersion function that is the inverse of the function of the stretcher. More precisely, the compressor also takes account of the dispersion compression that occurs in the various amplifiers. Just like the stretcher 3, the compressor 9 may be implemented using fiber components or a combination of bulk optical components such as prisms and diffraction gratings. In order to refine its compression, the compressor 9 may also contain bulk components, or indeed variable pitch (chirped) dielectric mirrors. The compressor 9 recompresses the amplified light pulses 17 in time in order to generate output laser pulses 20 that are amplified and time-compressed.

The power stage constitutes the core of the system since it needs to satisfy the following opposing criteria:
firstly, its architecture must limit the non-linear effects so as to make them as small as possible in order to avoid any time degradation of the sub-picosecond pulses. This tends to favor short lengths of fiber and low peak powers;
secondly, it must present gain that is flat over several tens of nanometers centered around 1590 nm. For Er-doped or Er—Yb doped amplifiers, this characteristic requires small mean population inversion in the fiber, and thus a long length of fiber in order to obtain sufficient gain.

The section of the amplifier optical fiber 7 in the power stage may be pumped directly in the doped core by a monomode laser instead of being pumped through the cladding. The pumping in the core serves to reduce the power needed for saturating the absorption of the pump. Furthermore, instead of using conventional laser diodes with pump wavelengths of 980 nm or 1480 nm, the wavelength λ_{P} of the pump 8 of the power amplifier fiber section is selected to lie in the range 1530 nm to 1565 nm. This pump wavelength λ_{P} serves advantageously to set the population inversion to a value that is constant along the fiber. This pump wavelength λ_{P} that is close to the emission wavelength also serves to reduce amplified spontaneous emission noise at a wavelength shorter than the pump wavelength λ_{P} and also significantly to increase the energy efficiency between pump and signal.

A pump operating at a wavelength λ_{P} close to 1550 nm may be made using a laser having an Er—Yb co-doped fiber.

It has been found that pumping the power amplifier stage in the 1530 nm-1565 nm band presents the major advantage of setting the population inversion to a value that is constant along the fiber. For a length of fiber that is sufficient for pumping at 1480 nm or 980 nm, it is also possible to obtain a spectral gain curve that is similar at the outlet from the fiber. Nevertheless, under such circumstances, the spectral dependency of the gain varies greatly along the fiber, with a maximum that goes from short wavelengths to high wavelengths. This gives rise to strong gain at the beginning of the fiber, followed by progressive filtering of short wavelengths. The result is that the peak power integrated over the length of the fiber, which determines the integral B responsible for non-linear effects, is three times greater for pumping at 1480 nm or at 980 nm than when pumping at 1550 nm. FIG. 2 shows two power curves for the output signal as a function of the length L_{F} of the section of power amplifier optical fiber 7, respectively for a conventional pump wavelength λ_{P} of 1480 nm and for a pump wavelength λ_{P} in accordance with the invention at 1550 nm. FIG. 2 shows the advantage of pumping the power amplifier stage at a wavelength λ_{P}≈1550 nm in order to minimize non-linear effects. For a fiber having a length L_{F} of about 10 meters (m) an equivalent output power is obtained from both curves, however the integral B of the curve corresponding to a pump wavelength λ_{P} of 1550 nm is visibly much smaller than the integral of the curve corresponding to a pump wavelength λ_{P} of 1480 nm.

The other significant advantage of pumping at about 1550 nm is the relative insensitivity of the shape of the spectral gain curve to variations in pump power and in fiber length, thereby contributing to the robustness of the system and making it easy to adjust the energy of the pulses without changing their spectral characteristics, as shown in FIG. 3. FIG. 3 plots spectral gain curves (α) obtained respectively for different section lengths L_{F} of power amplifier optical fiber 7 and for different pump wavelengths λ_{P}. The spectral gain curves (α) at a pump wavelength λ_{P} of 1550 nm are flat or almost flat over a wide spectral range, and their level increases as a function of length L_{F} of the amplifier fiber 7, unlike the spectral gain curves for a pump wavelength λ_{P} of 1480 nm. In a preferred embodiment, the section of amplifier optical fiber 7 is an optical fiber having a large effective area.

In a particularly advantageous embodiment, the amplifier fiber is pumped simultaneously by two pumps: one pump at 1550 nm and another pump at 980 nm. This combination of two pump wavelengths serves to obtain both strong amplification gain and weak non-linearities. It is observed that pumping at 1550 nm serves to eliminate the amplified spontaneous emission (ASE) generated by pumping at 980 nm, thereby increasing the energy of the output laser pulses at ≈1600 nm. This dual pumping serves to increase gain by 33% compared with single pump at 980 nm. It is thus possible to obtain 650 femtosecond (fs) laser pulses presenting energy of 2.2 μJ per pulse at a rate of 100 kHz.

Other characteristics of the invention make it possible to reduce non-linear effects so as to achieve the performance set out in Table 1.

TABLE 1
Operating ranges of the laser at the outputs of
the various components

| | | Pulse | | Pre- | Power | |
|---|---|---|---|---|---|---|
| | Oscillator | picker | Stretcher | amplifiers | amplifier | Compressor |
| Energy | ≈2 nJ | ≈1 nJ | ≈500 pJ | 10 nJ-20 nJ | 200 nJ-200 μJ | 100 nJ-100 μJ |
| Rate | ≈50 MHz | 10 kHz-1000 kHz | 10 kHz-1000 kHz | 10 kHz-1000 kHz | 10 kHz-1000 kHz | 10 kHz-1000 kHz |
| Mean | ≈100 mW | 10 μw-1000 μW | 5 μW-500 μW | 0.1 mW-20 mW | 2 mW-200 W | 1 mW-100 W |
| power | | | | | | |
| Peak | ≈20 kW | ≈10 kW | ≈1 W | 20 W-40 W | 400 W-400 kW | 50 kW-1 GW |
| power | | | | | | |
| Pulse | ≈100 fs | ≈100 fs | ≈500 ps | ≈500 ps | 500 ps | 100 fs-2000 fs |
| duration | | | | | | |

A laser 10 is obtained suitable for generating sub-picosecond laser pulses 20 of energy lying in the range 100 nJ to 100 μJ per pulse and of wavelengths situated in the range 1565 nm to 1625 nm. The characteristics of the laser of the invention enable the following performance to be achieved: rate lying in the range 10 kHz to 1 MHz, energy per pulse lying in the range 100 nJ to 100 μJ, pulse duration lying in the range 100 fs to 2 ps. The nominal performance is 500 fs pulses with energy per pulse of 5 μJ at a repetition frequency of 300 kHz.

The short pulse laser of the invention is for incorporating in an eye surgery system adapted to deep cutting of the cornea, in particular of pathological corneas, in order to perform partial or total transplants. This laser 10 is based essentially on optical fiber technology with the exception of the final compressor. The fiber amplifiers are based on erbium technology. The laser 10 emits in the wavelength range 1570 nm to 1610 nm, with the optimum being 1590 nm in order to benefit from a transparency window of the cornea while minimizing the impact of strong optical diffusion of pathological tissue.

A sub-picosecond laser is obtained at the wavelength of 1590 nm (±20 nm) that is adapted to rapidly cutting out corneas in depth. The laser is particularly adapted to pathological corneas that are generally diffusing corneas. The high wavelength of the radiation makes it possible to cut a zone of the cornea that is deep (1 millimeter (mm)) while suffering little diffusion. The pulses generated have a duration of less than 1 ps and energy lying in the range 100 nJ to 100 μJ, which corresponds to athermal cutting conditions. The laser has a repetition rate that is sufficient (≈100 kHz) for performing rapid cutting.

The elements constituting the laser of the invention are essentially based on optical fibers, thereby making the device robust, and enabling it to be incorporated easily in a medical environment. In the embodiment of the invention, the length L_{F} of optical fiber needed for power amplification is of the order of a few meters to about ten meters. In spite of this length, the laser of the invention enables non-linear effects in the power amplifier fiber to be limited.

A first application of the L-band high-energy sub-picosecond pulsed laser of the invention relates to an ophthalmic surgical tool for deep cutting of the cornea of the human or animal eye.

The laser of the invention enables the cornea of a patient to be cut rapidly and with good quality in order to treat the cornea or in order to perform a cornea transplant. The improvement in the quality of cutting makes it possible firstly to remove the cornea for treatment or for replacement more easily, and secondly it enables a new cornea to be re-implanted with better quality healing and with fewer complications after treatment or transplanting.

Another application of the invention in ophthalmic surgery is to provide a laser for treating glaucoma.

1. A chirped pulse amplification fiber laser (10) for amplifying sub-picosecond pulses of good spatial quality and high energy, the laser comprising:
an oscillator (1) suitable for emitting light pulses (11) of sub-picosecond duration and of emission wavelengths lying in the range 1565 nm to 1625 nm;
a fiber pulse stretcher (3) suitable for stretching the light pulses (11) in time;
one or more pre-amplification stages, each comprising a section of erbium-doped or erbium-ytterbium co-doped optical fiber (4) and a pump (6) suitable for optically pumping the section of pre-amplification optical fiber (4);
at least one rate reducer (2) suitable for reducing the repetition rate of the output laser pulses (20) to lie in the range 10 kHz to 1 MHz;
a power amplifier stage comprising a section of erbium-doped or erbium-ytterbium co-doped optical fiber (7) and a pump (8) suitable for optically pumping the section of power amplifier optical fiber (7), said power amplifier stage being suitable for producing amplified pulses (17);
a pulse compressor (9) suitable for recompressing the amplified light pulses (17) in time to produce output laser pulses (20);
the laser being characterized in that:
said pump (8) of the power amplifier optical fiber (7) is suitable for generating at least one pump wavelength λ_{P} lying in the range 1530 nm to 1565 nm so as to obtain output pulses (20) of emission wavelength lying in the range 1565 nm to 1625 nm, and of energy lying in the range 100 nJ to 100 μJ.

2. A pulsed laser (10) according to claim 1, characterized in that the section of optical fiber (7) of the power amplifier stage is a large mode area (LMA) fiber.

3. A pulsed laser according to claim 1, characterized in that the output beam has good spatial quality, with a coefficient M² less than 1.5.

4. A pulsed laser according to claim 1, characterized in that the pump (8) of the power amplifier is an erbium-doped or erbium-ytterbium co-doped optical fiber laser.

5. A pulsed laser according to claim 1, characterized in that the pump (8) of the power amplifier is directly coupled into the doped core of the power amplifier optical fiber (7).

6. A pulsed laser according to claim 1, characterized in that the pump (8) of the power amplifier is suitable for pumping the section of power amplifier optical fiber (7) in co-propagation and/or in contra-propagation manner.

7. A pulsed laser according to claim 1, characterized in that the pump (8) of the power amplifier is suitable for generating at least one second pump wavelength lying in the range 1530 nm to 1565 nm.

8. A pulsed laser according to claim 1, characterized in that the wavelength λ_{P} of the pump (8) of the power amplifier is adjustable so as to optimize the output pulses spectrally and temporally.

9. A pulsed laser according to claim 1, characterized in that it further includes another pump suitable for optically pumping the section of power amplifier optical fiber (7) at another pump wavelength λ′_{P} lying in the range 970 nm to 990 nm simultaneously with the first pump (8) at the pump wavelength λ_{P} so as to increase the amplification gain in the section of amplifier optical fiber.

10. A laser tool for ophthalmic surgery, the tool comprising an optical fiber pulsed laser according to claim 1.

11. A laser tool according to claim 10, characterized in that the energy of the laser pulses (20) is greater than 100 nJ.

12. A pulsed laser according to claim 2, characterized in that the output beam has good spatial quality, with a coefficient M² less than 1.5.
